(19) United States
(12) Patent Application Publication
(10) Pub. No.: US 2025/0123568 A1
(43) Pub. Date: Apr. 17, 2025

(54) COMPOSITION FOR REMOVING EDGE BEAD FROM METAL CONTAINING RESISTS, DEVELOPER COMPOSITION OF METAL CONTAINING RESISTS, AND METHOD OF FORMING PATTERNS USING THE COMPOSITION

(71) Applicant: SAMSUNG SDI CO., LTD., Yongin-si (KR)

(72) Inventors: Seung HAN, Suwon-si (KR); Hyungrang MOON, Suwon-si (KR); Taeho KIM, Suwon-si (KR); Jongpil HO, Suwon-si (KR); Jin-Hee BAE, Suwon-si (KR); Miyeon HAN, Suwon-si (KR)

(21) Appl. No.: 18/828,854

(22) Filed: Sep. 9, 2024

(30) Foreign Application Priority Data
Oct. 12, 2023 (KR) 10-2023-0136003

(51) Int. Cl. G03F 7/16; C07C 49/12; C07C 49/76; G03F 7/004

(52) U.S. Cl. CPC G03F 7/168; C07C 49/12; C07C 49/76; G03F 7/0042; G03F 7/162

(57) ABSTRACT

Provided are a composition selected from a composition for removing edge beads from metal-containing resists and a developer composition of metal-containing resists, and a method of forming patterns using the same, the composition includes a compound including at least two ketone groups; and an organic solvent including at least one selected from an acetate-based solvent and an alcohol-based solvent, wherein the compound including at least two ketone groups is included in an amount of about 10 to about 70 wt % based on the total weight of the composition, and the organic solvent is included in an amount of about 30 to about 90 wt % based on a total weight of the composition.

### CROSS-REFERENCE TO RELATED APPLICATION

The present application claims priority to and the benefit of Korean Patent Application No. 10-2023-0136003, filed on Oct. 12, 2023, in the Korean Intellectual Property Office, the entire content of which is hereby incorporated by reference.

### BACKGROUND

### 1. Field

Embodiments of this disclosure relate to compositions for removing edge beads from metal-containing resists and/or developer compositions of metal-containing resists, and methods of forming patterns using the same.

### 2. Description of the Related Art

In recent years, a semiconductor industry has been accompanied by a substantially continuous reduction of critical dimensions, and this dimensional reduction benefits from new types (or kinds) of high-performance photoresist materials and a patterning method that satisfy an interest in processing and patterning with increasingly smaller features.

With the recent rapid development of the semiconductor industry, a semiconductor device has increased an operation speed and large storage capacity, and in line with this improvement, process technology for improving integration, reliability, and a response speed of semiconductor devices is being developed. It is important to accurately control/implant impurities in working regions of a silicon substrate and to interconnect these regions to form a device and an ultra-high-density integrated circuit, which may be achieved by a photolithographic process. It is important to integrate the photolithographic process including coating a photoresist on the substrate, selectively exposing it to ultraviolet (UV) (including extreme ultraviolet (EUV)), electron beams, X rays, and/or the like, and then, developing it.

In the process of forming the photoresist layer, the resist is coated on the substrate, mainly while rotating the silicon substrate, wherein the resist is coated on an edge and rear surface of the substrate, which may cause pattern defects in the subsequent semiconductor processes such as etching and ion implantation processes. Accordingly, a process of stripping and removing the photoresist coated on the edge and rear surface of the silicon substrate by using a thinner composition, for example, an EBR (edge bead removal) process is performed. The EBR process utilizes a composition that exhibits excellent solubility for the photoresist and effectively removes beads and the photoresist remaining in the substrate and generates no or substantially no resist residue.

There is an interest in developing a photoresist that can ensure excellent etch resistance and resolution in the photolithography process, while improving sensitivity and CD (critical dimension) uniformity and can improve LER (line edge roughness) characteristics, and a developer composition that can implement it.

### SUMMARY

Some embodiments of the present disclosure provide a composition for removing edge beads from metal-containing resists and/or a developer composition of metal-containing resists.

Some embodiments provide a method of forming patterns using the composition.

A composition for removing edge beads from metal-containing resists and/or developer composition of metal-containing resists according to some embodiments includes a compound including at least two ketone groups; and an organic solvent including at least one selected from an acetate-based solvent and an alcohol-based solvent,
wherein the compound including at least two ketone groups is included in an amount of about 10 to about 70 wt % based on the total weight of the composition and the organic solvent is included in an amount of about 30 to about 90 wt % based on a total weight of the composition.

A method of forming patterns according to some embodiments includes coating a metal-containing resist composition on a substrate; coating a composition for removing edge beads from metal-containing resists along an edge of the substrate to remove a metal-containing resist edge bead; drying and heating to form a metal-containing photoresist film on the substrate; exposing the metal-containing photoresist film; and developing using a developer composition of metal-containing resists,

Wherein at least one selected from the composition for removing edge beads from metal-containing resists and the developer composition of metal-containing resists includes the aforementioned composition.

The composition for removing edge beads from the metal-containing resists according to some embodiments reduces the metal-based contamination inherent in the metal-containing resists and removes the resist coated on the edge and the rear surface of the substrate, thereby satisfying requirements of processing and patterning of smaller features.

The developer composition of metal-containing resists according to some embodiments provides excellent contrast characteristics, excellent sensitivity, and reduced line edge roughness (LER) by minimizing defects present in the metal-containing photoresist film after the exposure process and facilitating development.

### BRIEF DESCRIPTION OF THE DRAWINGS

The accompanying drawings, together with the specification, illustrate embodiments of the subject matter of the present disclosure, and, together with the description, serve to explain principles of embodiments of the subject matter of the present disclosure.

FIG. 1 is a schematic view of a photoresist coating apparatus.

FIGS. 2 to 4 are cross-sectional views showing a process sequence according to an embodiment of a method of forming patterns.

### DETAILED DESCRIPTION

Hereinafter, embodiments of the present disclosure are described in more detail with reference to the accompanying drawings. In the following description of the present disclosure, the well-known functions or constructions may not be described for reasons of clarity.

In order to clearly illustrate embodiments of the present disclosure, certain description and relationships may be omitted, and throughout the disclosure, the same or similar configuration elements are designated by the same reference numerals. Also, because the size and thickness of each configuration shown in the drawing may be arbitrarily shown for better understanding and ease of description, the present disclosure is not necessarily limited thereto.

In the drawings, the thickness of layers, films, panels, regions, etc., may be exaggerated for clarity. In the drawings, the thickness of a part of layers or regions, etc., may be exaggerated for clarity. It will be understood that when an element such as a layer, film, region, or substrate is referred to as being “on” another element, it can be directly on the other element or intervening elements may also be present.

In the present disclosure, the term “substituted” refers to replacement of a hydrogen atom by deuterium, a halogen, a hydroxy group, a thiol group, a cyano group, a carbonyl group, an amino group, a substituted or unsubstituted C1 to C30 amine group, a nitro group, a substituted or unsubstituted C1 to C40 silyl group, a C1 to C30 alkyl group, a C1 to C10 haloalkyl group, a C1 to C10 alkylsilyl group, a C3 to C30 cycloalkyl group, a C6 to C30 aryl group, a C1 to C20 alkoxy group, or a C1 to C20 sulfide group. The term “unsubstituted” means that a hydrogen atom remains as a hydrogen atom without being replaced by another substituent.

In the present disclosure, the term “alkyl group” means a linear or branched aliphatic hydrocarbon group, unless otherwise defined. The alkyl group may be a “saturated alkyl group” that does not contain any double or triple bonds.

The alkyl group may be a C1 to C20 alkyl group. In some embodiments, the alkyl group may be a C1 to C10 alkyl group or a C1 to C6 alkyl group. For example, a C1 to C5 alkyl group may have an alkyl chain that contains 1 to 5 carbon atoms, and may be selected from methyl, ethyl, propyl, iso-propyl, n-butyl, iso-butyl, sec-butyl, and t-butyl.

Examples of the alkyl group include a methyl group, an ethyl group, a propyl group, an isopropyl group, a butyl group, an isobutyl group, a tert-butyl group, a pentyl group, or a hexyl group, etc.

In the chemical formulas described herein, t-Bu refers to a tert-butyl group.

In the present disclosure, when a definition is not otherwise provided, the term “cycloalkyl group” refers to a monovalent cyclic aliphatic hydrocarbon group.

The cycloalkyl group may include a cyclopropyl group, a cyclobutyl group, a cyclopentyl group, a cyclohexyl group, etc.

The cycloalkyl group may be a C3 to C10 cycloalkyl group, for example, a C3 to C8 cycloalkyl group, a C3 to C7 cycloalkyl group, or a C3 to C6 cycloalkyl group. For example, the cycloalkyl group may be a cyclopropyl group, a cyclobutyl group, a cyclopentyl group, or a cyclohexyl group, but is not limited thereto.

In the present disclosure, when a definition is not otherwise provided, the “alkenyl group” may be a linear or branched aliphatic hydrocarbon group, and may refer to an aliphatic unsaturated alkenyl group containing one or more double bonds.

In the present disclosure, when a definition is not otherwise provided, the “alkynyl group” may be a linear or branched aliphatic hydrocarbon group, and may refer to an unsaturated alkynyl group containing one or more triple bonds.

In the present disclosure, the term “aryl group” means a substituent in which all elements of a cyclic substituent have p-orbitals, and these p-orbitals form a conjugate and may include monocyclic or polycyclic of fused ring (e.g., rings that share adjacent pairs of carbon atoms) functional groups.

In embodiments, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

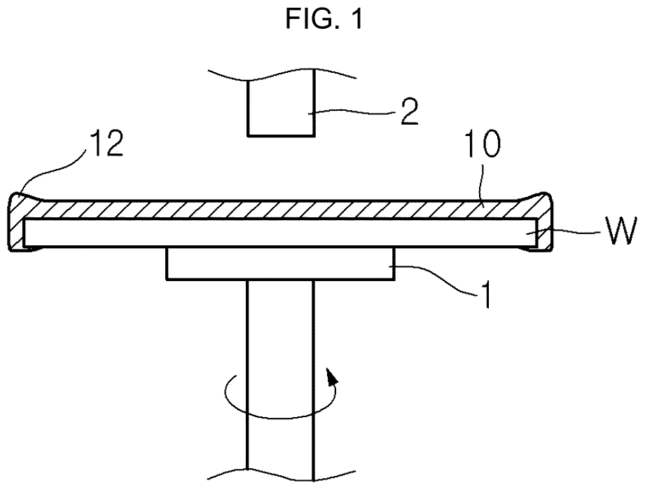

FIG. 1 is a schematic view showing the photoresist coating apparatus.

Referring to FIG. 1, a substrate support portion 1 on which a substrate W is placed is equipped therewith, and the substrate support portion 1 includes a spin chuck or a spin coater.

The substrate support portion 1 rotates in a first direction at a set or predetermined rotation speed to provide a centrifugal force to the substrate W. A spray nozzle 2 may be on or over the substrate support portion 1 but located off the upper portion of the substrate W in the stanby region so that the spray nozzle may be moved toward the upper portion of the substrate W and spray a photoresist solution 10 in the spraying step. For example, the spray nozzle 2 may be over and spaced apart from the substrate support portion 1, and the spray nozzle 2 may move toward or away from the substrate support portion 1. Accordingly, the photoresist solution 10 is coated on the surface of the substrate W by the centrifugal force. Herein, the photoresist solution 10 supplied to the center of the substrate W is coated while spreading to the edge of the substrate W by the centrifugal force, wherein a portion of photoresist solution 10 moves to the side surfaces of the substrate W and the lower surface of the edge of the substrate.

In embodiments, in the coating process, the photoresist solution 10 is coated mainly in a spin coating method, wherein a set or predetermined amount of the photoresist solution 10 having viscosity (e.g., a set viscosity) is supplied to the center portion of the substrate W and gradually spreads toward the edge of the substrate W by the centrifugal force.

Accordingly, the photoresist film is evenly formed by a rotational speed of the substrate support portion.

This rotation evaporates a solvent from the photoresist solution 10 and thereby gradually increases the viscosity of the photoresist solution 10, resulting in making a relatively large amount of the photoresist solution 10 accumulate on the edge of the substrate by the action of surface tension and severely even onto the lower surface of the edge of the substrate, these accumulations of the photoresist solution 10 are referred to as edge beads 12.

Hereinafter, a composition for removing edge beads from metal-containing resists and/or a developer composition of metal-containing resists according to some embodiments is described.

The composition for removing edge beads from metal-containing resists and/or developer composition of metal-containing resists according to some embodiments includes a compound including at least two ketone groups; and
an organic solvent including at least one selected from an acetate-based solvent and an alcohol-based solvent, wherein the compound including at least two ketone groups is included in an
amount of about 10 to about 70 wt % based on the total weight of the composition and the organic solvent is included in an amount of about 30 to about 90 wt % based on a total weight of the composition.

The compound including at least two ketone groups can adjust the sensitivity so that contrast is exhibited for a photoresist film in a low exposure energy state, and thus, can further increase the difference in solubility between an exposed region and an unexposed region.

Accordingly, the occurrence of scum and/or residual film in the unexposed region can be suppressed or reduced.

For example, the compound including at least two ketone groups may be included in an amount of about 10 to about 60 wt % based on a total weight of the composition.

For example, the compound including at least two ketone groups may be included in an amount of about 10 to about 50 wt % based on a total weight of the composition.

In some embodiments, the compound including at least two ketone groups may include at least one halogen.

The compound including at least two ketone groups may be represented by Chemical Formula 1 or Chemical Formula 2.

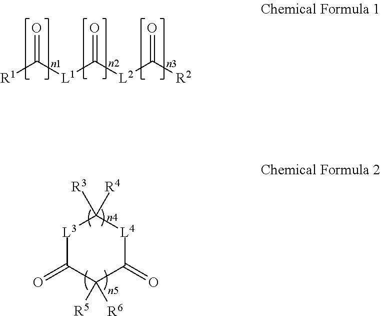

In Chemical Formula 1 and Chemical Formula 2,
R¹ to R⁶ are each independently hydrogen, a halogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof,
L¹ to L⁴ are each independently a single bond (e.g., a single covalent bond), a carbonyl group, substituted or unsubstituted C1 to C20 alkylene group, or a combination thereof,
n1 to n5 are each independently one of integers of 0 to 2,
n1+n2+n3 is 2 or more, and
n4+n5 is 1 or more.

In Chemical Formula 1, at least one selected from R¹, R², L¹, and L² may be a halogen, a C1 to C20 alkyl group substituted with at least one halogen, a C3 to C20 cycloalkyl group substituted with at least one halogen, a C2 to C20 alkenyl group substituted with at least one halogen, a C2 to C20 alkynyl group substituted with at least one halogen, a C6 to C30 aryl group substituted with at least one halogen, a C1 to C20 alkylene group substituted with at least one halogen, or a combination thereof, and
in Chemical Formula 2, at least one selected from R³ to R⁶, L³, and L⁴ may be a halogen, a C1 to C20 alkyl group substituted with at least one halogen, a C3 to C20 cycloalkyl group substituted with at least one halogen, a C2 to C20 alkenyl group substituted with at least one halogen, a C2 to C20 alkynyl group substituted with at least one halogen, a C6 to C30 aryl group substituted with at least one halogen, a C1 to C20 alkylene group substituted with at least one halogen, or a combination thereof.

In some embodiments, the compound including at least two ketone groups may include at least one selected from fluoro and chloro. As used herein, “at least one selected from fluoro and chloro” may mean at least one selected from fluorine and chlorine (e.g., at least one selected from a fluorine atom and a chlorine atom).

In some embodiments,

In Chemical Formula 1, at least one selected from R¹, R², L¹, and L² may be fluoro; chloro; a methyl group substituted with at least one selected from fluoro and chloro; an ethyl group substituted with at least one selected from fluoro and chloro; a propyl group substituted with at least one selected from fluoro and chloro; a butyl group substituted with at least one selected from fluoro and chloro; an isopropyl group substituted with at least one selected from fluoro and chloro; a tert-butyl group substituted with at least one selected from fluoro and chloro; a 2,2-dimethylpropyl group substituted with at least one selected from fluoro and chloro; a tert-pentyl group substituted with at least one selected from fluoro and chloro; a cyclopropyl group substituted with at least one selected from fluoro and chloro; a cyclobutyl group substituted with at least one selected from fluoro and chloro; a cyclopentyl group substituted with at least one selected from fluoro and chloro; a cyclohexyl group substituted with at least one selected from fluoro and chloro; an ethenyl group substituted with at least one selected from fluoro and chloro; a propenyl group substituted with at least one selected from fluoro and chloro; a butenyl group substituted with at least one selected from fluoro and chloro; an ethynyl group substituted with at least one selected from fluoro and chloro; a propynyl group substituted with at least one selected from fluoro and chloro; a butynyl group substituted with at least one selected from fluoro and chloro; a phenyl group substituted with at least one selected from fluoro and chloro; a tolyl group substituted with at least one selected from fluoro and chloro; a xylene group substituted with at least one selected from fluoro and chloro; a methylene group substituted with at least one selected from fluoro and chloro; an ethylene group substituted with at least one selected from fluoro and chloro; a propylene group substituted with at least one selected from fluoro and chloro, or a combination thereof, and
in Chemical Formula 2, at least one selected from R³ to R⁶, L³, and L⁴ may be fluoro; chloro; a methyl group substituted with at least one selected from fluoro and chloro; an ethyl group substituted with at least one selected from fluoro and chloro; a propyl group substituted with at least one selected from fluoro and chloro; a butyl group substituted with at least one selected from fluoro and chloro; an isopropyl group substituted with at least one selected from fluoro and chloro; a tert-butyl group substituted with at least one selected from fluoro and chloro; a 2,2-dimethylpropyl group substituted with at least one selected from fluoro and chloro; a tert-pentyl group substituted with at least one selected from fluoro and chloro; a cyclopropyl group substituted with at least one selected from fluoro and chloro; a cyclobutyl group substituted with at least one selected from fluoro and chloro; a cyclopentyl group substituted with at least one selected from fluoro and chloro; a cyclohexyl group substituted with at least one selected from fluoro and chloro; an ethenyl group substituted with at least one selected from fluoro and chloro; a propenyl group substituted with at least one selected from fluoro and chloro; a butenyl group substituted with at least one selected from fluoro and chloro; an ethynyl group substituted with at least one selected from fluoro and chloro; a propynyl group substituted with at least one selected from fluoro and chloro; a butynyl group substituted with at least one selected from fluoro and chloro; a phenyl group substituted with at least one selected from fluoro and chloro; a tolyl group substituted with at least one selected from fluoro and chloro; a xylene group substituted with at least one selected from fluoro and chloro; a methylene group substituted with at least one selected from fluoro and chloro; an ethylene group substituted with at least one selected from fluoro and chloro; a propylene group substituted with at least one selected from fluoro and chloro, or a combination thereof.

For example, the compound including at least two ketone groups may include at least one fluoro (e.g., at least one fluorine atom).

For example, the compound including at least two ketone groups may include at least one chloro (e.g., at least one chlorine atom).

The organic solvent included in the composition for removing edge beads from metal-containing resists and/or developer composition according to some embodiments may include at least one selected from an acetate based solvent and an alcohol based solvent, for example, propylene glycol methyl ether (PGME), propylene glycol methyl ether acetate (PGMEA), propylene glycol butyl ether (PGBE), ethylene glycol methyl ether, diethylglycolethylmethylether, dipropylglycoldimethylether, ethanol, 2-butoxyethanol, n-propanol, isopropanol, n-butanol, isobutanol, hexanol, ethylene glycol, propylene glycol, ethyl acetate, methyl 3-methoxypropionate, ethyl 3-ethoxypropionate, diacetone alcohol, cyclohexanone, methyl-2-hydroxy-2-methylpropionate (HBM), gamma butyrolactone (GBL), 1-butanol (n-butanol), ethyllactate (EL), butyllactate (n-butylactate), 4-methyl-2-pentenol (or, methyl isobutyl carbinol (MIBC)), 1-methoxy-2-propanol, 1-ethoxy-2-propanol, 2-hydroxyethyl propionate, 2-hydroxy-2-methylethyl propionate, ethoxyethyl acetate, hydroxyethyl acetate, 2-hydroxy-3-methylbutanoic acid, 3-methoxymethyl propionate, 3-methoxyethyl propionate, 3-ethoxyethyl propionate, 3-ethoxymethyl propionate, methyl pyruvate, ethyl pyruvate, butyl acetate, n-butyl acetate, 1-methoxy-2-propyl acetate, methoxyethoxy propionate, ethoxyethoxypropionate, or a mixture thereof, but is not limited thereto.

The composition for removing edge beads from metal-containing resists according to the present disclosure may be particularly effective in removing metal-containing resists, and, for example, undesirable metal residues, such as tin-based metal residues.

In embodiments, the developer composition of metal-containing resists according to the present disclosure minimizes or reduces defects present in the metal-containing photoresist film after the exposure process and allows for easy development, thereby realizing excellent pattern characteristics.

In embodiments, excellent sensitivity and reduced line edge roughness (LER) can be achieved.

The composition for removing edge beads from metal-containing resists and/or developer composition of metal-containing resists may further include an acid compound such as carboxylic acid, phosphoric acid, phosphorous acid, sulfuric acid, and/or the like.

In embodiments including other additives to be further described below, the organic solvent may be included in a balance amount excluding the included components.

The composition for removing edge beads from metal-containing resists and/or developer composition of metal-containing resists may further include at least one other additive selected from a surfactant, a dispersant, a moisture absorbent, and a coupling agent.

According to some example embodiments, a method of forming patterns includes removing the edge beads using the aforementioned composition for removing edge beads from the metal-containing resist. For example, the manufactured pattern may be a photoresist pattern. In embodiments, the photoresist pattern may be a negative-type photoresist pattern.

A method of forming patterns according to some embodiments includes coating a metal-containing resist composition on a substrate, coating the aforementioned composition for removing edge beads from metal-containing resists along an edge of the substrate, drying and heating to form a metal-containing photoresist film on the substrate, exposing the metal-containing photoresist film, and developing it.

For example, the forming of patterns using the metal-containing resist composition may include coating a metal-containing resist composition on a substrate on which a thin film is formed by spin coating, slit coating, inkjet printing, etc., and drying the coated metal-containing resist composition to form a photoresist film. The metal-containing resist composition may include a tin-based compound, for example, the tin-based compound may include at least one selected from an organooxy group-containing tin compound and an organocarbonyloxy group-containing tin compound.

In embodiments, the composition for removing edge beads from the metal-containing resists along the edge of the substrate may be coated while rotating the substrate at a suitable or appropriate speed (e.g., 500 rpm or more).

Subsequently, a first heat treatment process of heating the substrate on which the photoresist film is formed is performed. The first heat treatment process may be performed at a temperature of about 80° C. to about 120° C. and in this process, the solvent is evaporated and the photoresist film may be more firmly adhered to the substrate.

Then, the photoresist film is selectively exposed.

Examples of light that may be used in the exposure process may include not only light having a short wavelength such as i-line (wavelength 365 nm), KrF excimer laser (wavelength 248 nm), and/or ArF excimer laser (wavelength 193 nm), but also light having a high energy wavelength such as EUV (Extreme UltraViolet) light, wavelength 13.5 nm), E-Beam (electron beam), etc.

In embodiments, the light for exposure according to some example embodiments may be short-wavelength light having light in a wavelength range of about 5 nm to about 150 nm, and light having a high energy wavelength such as EUV (Extreme UltraViolet, wavelength 13.5 nm), E-Beam (electron beam), etc.

In the forming the photoresist pattern, a negative-type pattern may be formed.

The exposed region of the photoresist film has a solubility different from that of the unexposed region of the photoresist film as a polymer is formed by a crosslinking reaction such as a condensation reaction between organometallic compounds.

Then, a second heat treatment process is performed on the substrate. The second heat treatment process may be performed at a temperature of about 90° C. to about 200° C. By performing the second heat treatment process, the exposed region of the photoresist film becomes difficult to be dissolved in a developer.

For example, the photoresist pattern corresponding to the negative tone image may be completed by dissolving and removing the photoresist film corresponding to the unexposed region using an organic solvent such as 2-heptanone.

The developer used in the method of forming patterns according to some embodiments may be an organic solvent, for example, ketones such as methyl ethyl ketone, acetone, cyclohexanone, and/or 2-heptanone, alcohols such as 4-methyl-2-propanol, 1-butanol, isopropanol, 1-propanol, and/or methanol, esters such as propylene glycol monomethyl ether acetate, ethyl acetate, ethyl lactate, n-butyl acetate, butyrolactone, aromatic compounds such as benzene, xylene, and/or toluene, or a combination thereof.

As described above, the photoresist pattern formed by exposure to not only light having a short wavelength such as i-line (wavelength 365 nm), KrF excimer laser (wavelength 248 nm), and/or ArF excimer laser (wavelength 193 nm), but also light having a high energy such as EUV (Extreme UltraViolet; wavelength of 13.5 nm), and an E-beam (electron beam) may have a thickness width of about 5 nm to about 100 nm. For example, the photoresist pattern may be formed to have a thickness width of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm.

In embodiments, the photoresist pattern may have a pitch having a half-pitch of less than or equal to about 50 nm, for example less than or equal to 40 nm, for example less than or equal to 30 nm, for example less than or equal to 20 nm, for example less than or equal to 15 nm and a line width roughness of less than or equal to about 10 nm, less than or equal to about 5 nm, less than or equal to about 3 nm, or less than or equal to about 2 nm.

A method of forming patterns according to some embodiments includes coating a metal-containing resist composition on a substrate, removing edge beads of the metal-containing resists, drying and heating to form a metal-containing resist film on the substrate, exposing the metal-containing photoresist film, and developing using the aforementioned developer composition of metal-containing resists.

The coating of the metal-containing resist composition on a substrate is the same as described above.

The removing of edge beads of the metal-containing resists may be performed by coating a suitable or appropriate amount of any suitable organic solvent and/or composition for removing edge beads along the edge of the substrate while spinning the substrate at a suitable or appropriate speed (e.g., 500 rpm or more).

The drying and heating to form a metal-containing resist film on the substrate is the same as described above.

The exposing of the metal-containing photoresist film is the same as described above.

The photoresist pattern corresponding to the negative tone image may be completed by dissolving the photoresist film corresponding to the unexposed region using the aforementioned developer composition of metal-containing resists and then removing the photoresist film.

The metal compound included in the metal-containing resist composition may include at least one selected from an organic oxy group and an organic carbonyloxy group.

As an example, the metal compound included in the metal-containing resist composition may be represented by Chemical Formula 3.

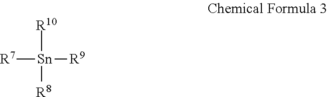

In Chemical Formula 3,
R⁷ is selected from a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and L^{a}-O—R^{a} (wherein L^{a} is a substituted or unsubstituted C1 to C20 alkylene group, R^{a} is a substituted or unsubstituted C1 to C20 alkyl group or a substituted or unsubstituted C6 to C20 aryl group),
R⁸ to R¹⁰ are each independently a halogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, alkoxy or aryloxy (—OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl group (—O(CO)R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), alkylamido or dialkylamido (—NR^{d}R^{e}, wherein R^{d} and R^{e} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), amidato (—NR^{f}(COR^{g}), wherein R^{f} and R^{g} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or amidinato (—NR^{h}C(NRⁱ)R^{j}, wherein R^{h}, Rⁱ, and R^{j} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), and

At least one selected from R⁸ to R¹⁰ are each independently a halogen, alkoxy or aryloxy (—OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl group (—O(CO) R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), alkylamido or dialkylamido (—NR^{d}R^{e}, wherein R^{d} and R^{e} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), amidato (—NR^{f}(COR^{g}), wherein R^{f} and R^{g} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or amidinato (—NR^{h}C(NRⁱ) Rⁱ, wherein R^{h}, Rⁱ, and Rⁱ are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).

For example, the metal compound included in the metal-containing resist composition may include at least one selected from an alkyloxy group and an alkylcarbonyloxy group.

Hereinafter, a method of forming patterns by development is described in detail with reference to the drawings.

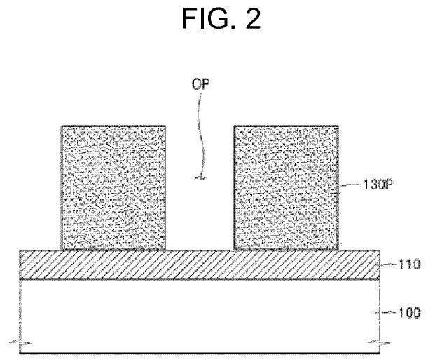

FIGS. 2-4 are cross-sectional views illustrating a process sequence in order to explain a method of forming patterns according to embodiments of the present disclosure.

Referring to FIG. 2, the exposed photoresist film is developed to form a photoresist pattern 130P on the substrate 100.

In some embodiments, the exposed photoresist film may be developed to remove an unexposed region of the photoresist film, and the photoresist pattern 130P including the exposed region of the photoresist film may be formed. The photoresist pattern 130P may include a plurality of openings OP.

In some embodiments, the development of the photoresist film may be performed through an NTD (negative-tone development) process. Herein, the metal-containing photoresist developer composition according to some embodiments may be used as a developer composition.

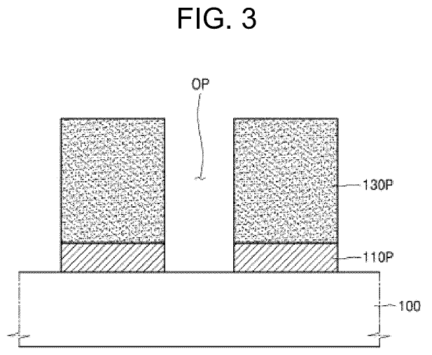

Referring to FIG. 3, the photoresist pattern 130P is used to process a feature layer 110 in the result of FIG. 2.

For example, the feature layer 110 is processed through various suitable processes of etching a feature layer 110 exposed through the openings OP of the photoresist pattern 130P, injecting impurity ions into the feature layer 110, forming an additional film on the feature layer 110 through the openings OP, deforming a portion of the feature layer 110 through the openings OP, and/or the like. FIG. 3 illustrates an example process of processing a feature pattern 110P by etching the feature layer 110 exposed through the openings OP.

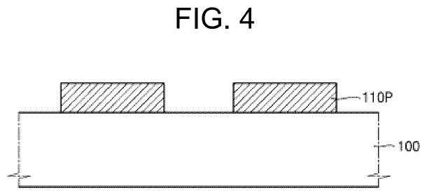

Referring to FIG. 4, the photoresist pattern 130P remaining on the feature pattern 110P is removed in the result of FIG. 3. In order to remove the photoresist pattern 130P, an ashing and/or stripping process may be used.

A method of forming patterns according to some embodiments includes coating a metal-containing resist composition on a substrate, coating the aforementioned composition for removing edge beads from metal-containing resists along an edge of the substrate, drying and heating to form a metal-containing photoresist film on the substrate, exposing the metal-containing photoresist film, and developing using the aforementioned developer composition of metal-containing resists.

The method is specifically the same as described above, but in the removing of edge beads and the developing, the composition for removing edge beads from metal-containing resists and/or the developer composition of metal-containing resists according to the present disclosure are concurrently (e.g., simultaneously) used to effectively improve an effect of removing the edge beads and solubility of the unexposed region and thereby improve processing and patterning of smaller features, resultantly realizing excellent contrast characteristics, excellent sensitivity, and reduced line edge roughness (LER).

Hereinafter, embodiments of the present disclosure will be described in more detail through examples relating to the preparation of the aforementioned composition for removing edge beads from metal-containing resists and developer composition of metal-containing resists. However, the technical features of the present disclosure are not limited by the following examples.

### Preparation of Composition for Removing Edge Beads from Metal-containing Resists/Developer Composition

### Examples 1 to 6 and Comparative Examples 1 to 5

One type among acetylacetone (AcAc), 1,1,1,5,5,5-Hexafluoro-2,4-pentanedione (Hfac), 3-chloro-2,4-pentanedione (Cl—AcAc), formic acid (FA), and acetic acid (AA) was mixed together with propylene glycol methyl ether acetate (PGMEA) as a solvent in each composition shown in Table 1 and then, completely dissolved by shaking at room temperature (25° C.). Thereafter, the final composition was obtained by passing through a filter made of PTFE having a pore size of 1 μm.

TABLE 1

| | Composition for removing edge | |
|---|---|---|
| | beads from metal-containing | |
| | resists/developer composition | |
| | organic solvent | ketone group-containing |
| | (wt %) | compound (wt %) |
| Example 1 | PGMEA 80 | AcAc 20 |
| Example 2 | PGMEA 60 | AcAc 40 |
| Example 3 | PGMEA 80 | Hfac 20 |
| Example 4 | PGMEA 60 | Hfac 40 |
| Example 5 | PGMEA 80 | Cl-AcAc 20 |
| Example 6 | PGMEA 60 | Cl-AcAc 40 |
| Comparative Example 1 | PGMEA 100 | — |
| Comparative Example 2 | PGMEA 98 | FA 2.0 |
| Comparative Example 3 | PGMEA 98 | AA 2.0 |
| Comparative Example 4 | PGMEA 92 | AcAc 8.0 |
| Comparative Example 5 | PGMEA 20 | AcAc 80 |

### Preparation of Organometal-Containing Photoresist Composition

Photoresist compositions were prepared by dissolving an organometallic compound containing a structural unit represented by Chemical Formula C in 4-methyl-2-pentanol at a concentration of 1 wt % and filtering the solution with a 0.1 μm PTFE syringe filter.

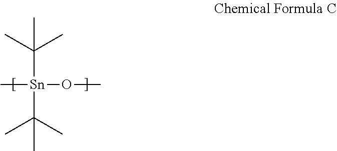

### Evaluation 1: Evaluation of Contrast Performance and Sensitivity

Each of the manufactured metal-containing photoresist (PR) compositions was spin-coated on an 8-inch wafer at 1,500 rpm for 30 seconds and then, heat-treated at 130° C. for 60 seconds to manufacture a coated wafer.

The coated wafer was exposed with a rectangle pattern with a size of 1.2 cm×0.9 cm to a dose of 10 to 100 mJ by using a KrF scanner (PAS 5500/700D, ASML), heat-treated at 180° C. for 60 seconds, developed by respectively applying the developer compositions according to Examples 1 to 6 and Comparative Examples 1 to 5, and finally heat-treated at 200° C. for 60 seconds, thereby completing patterned wafers. The patterned wafers were measured with respect to a thickness of each exposed region to obtain a contrast curve, from which contrast performance (γ(contrast)) was calculated, and the results are shown in Table 2.

γ(contrast)=1/log (D₁₀₀/D₀)
D₁₀₀=An exposure dose at which PR began to remain 100%
D₀=An exposure dose at which PR was completely removed

### Evaluation 2: Evaluation of Sensitivity and Line Edge Roughness (LER)

A linear array of 50 circular pads having a diameter of 500 μm was projected onto a wafer by using EUV light (Lawrence Berkeley National Laboratory Micro Exposure Tool, MET). Herein, exposure time of the pads was adjusted, so that an incremental EUV dose was applied to each pad.

Subsequently, a resist and a substrate therefrom were exposed at 160° C. for seconds on a hot plate and then, post-exposure baked (PEB). The baked film was immersed in the developer according to the examples and comparative examples for seconds, respectively, and then a negative tone image was formed. Finally, the process was terminated by baking the film at 150° C. for 2 minutes on the hot plate. After measuring sensitivity and LER from the FE-SEM image, sensitivity and line edge roughness were evaluated according to the following criteria, and the results are shown in Table 2.

### Sensitivity Evaluation Criteria

A: less than 16 mJ/cm²
B: greater than or equal to 16 mJ/cm²

### LER Evaluation Criteria

∘: less than or equal to 2 nm
Δ: greater than 2 nm and less than or equal to 5 nm
X: greater than 5 nm

TABLE 2

| | γ (contrast) | Sensitivity | LER |
|---|---|---|---|
| Example 1 | 35 | A | ◯ |
| Example 2 | 31 | A | ◯ |
| Example 3 | 34 | A | ◯ |
| Example 4 | 31 | A | ◯ |
| Example 5 | 36 | A | ◯ |
| Example 6 | 32 | A | ◯ |
| Comparative Example 1 | 12 | A | X |
| Comparative Example 2 | 24 | B | Δ |
| Comparative Example 3 | 20 | B | Δ |
| Comparative Example 4 | 38 | A | Δ |
| Comparative Example 5 | 26 | B | ◯ |

### * HP: Half Pitch Reference

Referring to Table 2, applying the metal-containing photoresist developer compositions according to the examples, compared with applying the metal-containing photoresist developer compositions according to the comparative examples, provided excellent contrast performance and excellent sensitivity characteristics and realized reduced line edge roughness.

Hereinbefore, certain embodiments of the present disclosure have been described and illustrated, however, it should be apparent to a person having ordinary skill in the art that the present disclosure is not limited to the embodiment as described, and may be variously modified and transformed without departing from the spirit and scope of the present disclosure. Accordingly, the modified or transformed embodiments as such may not be understood separately from the technical ideas and aspects of the present disclosure, and the modified embodiments are within the scope of the appended claims of the present disclosure, and equivalents thereof.

### DESCRIPTION OF SYMBOLS

1: substrate support portion 2: spray nozzle
10: photoresist solution 12: edge bead
100: substrate OP: opening
110: feature layer
110P: feature pattern 130P: photoresist pattern

What is claimed is:

1. A composition selected from a composition for removing edge beads from metal-containing resists and a developer composition of metal-containing resists, each comprising:
a compound comprising at least two ketone groups; and
an organic solvent comprising at least one selected from an acetate-based solvent and an alcohol-based solvent,
wherein the compound comprising at least two ketone groups is included in an amount of about 10 to about 70 wt % based on the total weight of the composition and the organic solvent is included in an amount of about 30 to about 90 wt % based on a total weight of the composition.

2. The composition as claimed in claim 1, wherein:
the compound comprising at least two ketone groups is included in an amount of about 10 to about 60 wt % based on a total weight of the composition.

3. The composition as claimed in claim 1, wherein:
the compound comprising at least two ketone groups is included in an amount of about 10 to about 50 wt % based on a total weight of the composition.

4. The composition as claimed in claim 1, wherein:
the compound comprising at least two ketone groups comprises at least one halogen.

5. The composition as claimed in claim 1, wherein:
the compound comprising at least two ketone groups is represented by Chemical Formula 1 or Chemical Formula 2: 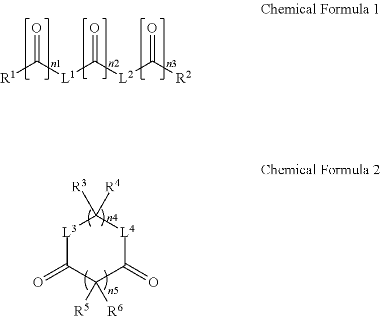
wherein, in Chemical Formula 1 and Chemical Formula 2,
R¹ to R⁶ are each independently hydrogen, a halogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof,
L¹ to L⁴ are each independently a single bond, a carbonyl group, substituted or unsubstituted C1 to C20 alkylene group, or a combination thereof,
n1 to n5 are each independently one of integers of 0 to 2,
n1+n2+n3 is 2 or more, and
n4+n5 is 1 or more.

6. The composition as claimed in claim 5, wherein:
in Chemical Formula 1, at least one selected from R¹, R², L¹, and L² is a halogen, a C1 to C20 alkyl group substituted with at least one halogen, a C3 to C20 cycloalkyl group substituted with at least one halogen, a C2 to C20 alkenyl group substituted with at least one halogen, a C2 to C20 alkynyl group substituted with at least one halogen, a C6 to C30 aryl group substituted with at least one halogen, a C1 to C20 alkylene group substituted with at least one halogen, or a combination thereof, and
in Chemical Formula 2, at least one selected from R³ to R⁶, L³, and L⁴ is a halogen, a C1 to C20 alkyl group substituted with at least one halogen, a C3 to C20 cycloalkyl group substituted with at least one halogen, a C2 to C20 alkenyl group substituted with at least one halogen, a C2 to C20 alkynyl group substituted with at least one halogen, a C6 to C30 aryl group substituted with at least one halogen, a C1 to C20 alkylene group substituted with at least one halogen, or a combination thereof.

7. The composition as claimed in claim 5, wherein:
in Chemical Formula 1, at least one selected from R¹, R², L¹, and L² is fluoro; chloro; a methyl group substituted with at least one selected from fluoro and chloro; an ethyl group substituted with at least one selected from fluoro and chloro; a propyl group substituted with at least one selected from fluoro and chloro; a butyl group substituted with at least one selected from fluoro and chloro; an isopropyl group substituted with at least one selected from fluoro and chloro; a tert-butyl group substituted with at least one selected from fluoro and chloro; a 2,2-dimethylpropyl group substituted with at least one selected from fluoro and chloro; a tert-pentyl group substituted with at least one selected from fluoro and chloro; a cyclopropyl group substituted with at least one selected from fluoro and chloro; a cyclobutyl group substituted with at least one selected from fluoro and chloro; a cyclopentyl group substituted with at least one selected from fluoro and chloro; a cyclohexyl group substituted with at least one selected from fluoro and chloro; an ethenyl group substituted with at least one selected from fluoro and chloro; a propenyl group substituted with at least one selected from fluoro and chloro; a butenyl group substituted with at least one selected from fluoro and chloro; an ethynyl group substituted with at least one selected from fluoro and chloro; a propynyl group substituted with at least one selected from fluoro and chloro; a butynyl group substituted with at least one selected from fluoro and chloro; a phenyl group substituted with at least one selected from fluoro and chloro; a tolyl group substituted with at least one selected from fluoro and chloro; a xylene group substituted with at least one selected from fluoro and chloro; a methylene group substituted with at least one selected from fluoro and chloro; an ethylene group substituted with at least one selected from fluoro and chloro; a propylene group substituted with at least one selected from fluoro and chloro, or a combination thereof, and
in Chemical Formula 2, at least one selected from R³ to R⁶, L³, and L⁴ is fluoro; chloro; a methyl group substituted with at least one selected from fluoro and chloro; an ethyl group substituted with at least one selected from fluoro and chloro; a propyl group substituted with at least one selected from fluoro and chloro; a butyl group substituted with at least one selected from fluoro and chloro; an isopropyl group substituted with at least one selected from fluoro and chloro; a tert-butyl group substituted with at least one selected from fluoro and chloro; a 2,2-dimethylpropyl group substituted with at least one selected from fluoro and chloro; a tert-pentyl group substituted with at least one selected from fluoro and chloro; a cyclopropyl group substituted with at least one selected from fluoro and chloro; a cyclobutyl group substituted with at least one selected from fluoro and chloro; a cyclopentyl group substituted with at least one selected from fluoro and chloro; a cyclohexyl group substituted with at least one selected from fluoro and chloro; an ethenyl group substituted with at least one selected from fluoro and chloro; a propenyl group substituted with at least one selected from fluoro and chloro; a butenyl group substituted with at least one selected from fluoro and chloro; an ethynyl group substituted with at least one selected from fluoro and chloro; a propynyl group substituted with at least one selected from fluoro and chloro; a butynyl group substituted with at least one selected from fluoro and chloro; a phenyl group substituted with at least one selected from fluoro and chloro; a tolyl group substituted with at least one selected from fluoro and chloro; a xylene group substituted with at least one selected from fluoro and chloro; a methylene group substituted with at least one selected from fluoro and chloro; an ethylene group substituted with at least one selected from fluoro and chloro; a propylene group substituted with at least one selected from fluoro and chloro, or a combination thereof.

8. The composition as claimed in claim 1, wherein:
the composition further comprises an acid compound of carboxylic acid, phosphoric acid, phosphorous acid, sulfuric acid, or a combination thereof.

9. A method of forming patterns, comprising:
coating a metal-containing resist composition on a substrate;
coating a composition for removing edge beads from metal-containing resists along an edge of the substrate to remove a metal-containing resist edge bead;
drying and heating to form a metal-containing photoresist film on the substrate;
exposing the metal-containing photoresist film; and
developing using a developer composition of metal-containing resists,
wherein at least one selected from the composition for removing edge beads from metal-containing resists and the developer composition of metal-containing resists are each the composition as claimed in claim 1.

10. The method as claimed in claim 9, wherein:
a metal compound included in the metal-containing resist composition comprises at least one selected from an organic oxy group and an organic carbonyloxy group.

11. The method as claimed in claim 9, wherein:
a metal compound included in the metal-containing resist composition is represented by Chemical Formula 3: 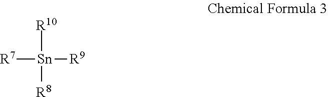
wherein, in Chemical Formula 3,
R⁷ is selected from a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, and L^{a}-O—R^{a} (wherein L^{a} is a substituted or unsubstituted C1 to C20 alkylene group and R^{a} is a substituted or unsubstituted C1 to C20 alkyl group or a substituted or unsubstituted C6 to C20 aryl group),
R⁸ to R¹⁰ are each independently a halogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, an alkoxy group or an aryloxy group (—OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl group (—O(CO) R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylamido group or a dialkylamido group (—NR^{d}R^{e}, wherein R^{d} and R^{e} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidato group (—NR^{f}(COR^{g}), wherein R^{f} and R^{g} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or an amidinato group (—NR^{h}C(NRⁱ) Rⁱ, wherein R^{h}, Rⁱ, and Rⁱ are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof)
at least one selected from R⁸ to R¹⁰ is a halogen, an alkoxy group or an aryloxy group (—OR^{b}, wherein R^{b} is a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), a carboxyl group (—O(CO) R^{c}, wherein R^{c} is hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an alkylamido group or a dialkylamido group (—NR^{d}R^{e}, wherein R^{d} and R^{e} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), an amidato group (—NR^{f}(COR^{g}), wherein R^{f} and R^{g} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof), or an amidinato group (—NR^{h}C(NRⁱ)R^{j}, wherein R^{h}, Rⁱ, and R^{j} are each independently hydrogen, a substituted or unsubstituted C1 to C20 alkyl group, a substituted or unsubstituted C3 to C20 cycloalkyl group, a substituted or unsubstituted C2 to C20 alkenyl group, a substituted or unsubstituted C2 to C20 alkynyl group, a substituted or unsubstituted C6 to C30 aryl group, or a combination thereof).
